(19) United States
(12) Patent Application Publication
(10) Pub. No.: US 2026/0244817 A1
(43) Pub. Date: Aug. 20, 2026

(54) APPARATUS, METHOD, AND PROGRAM

(71) Applicant: Yokogawa Electric Corporation, Tokyo (JP)

(72) Inventors: Ayumu SAKAKI, Tokyo (JP); Tetsushi NAMATAME, Tokyo (JP)

(21) Appl. No.: 19/118,350

(22) PCT Filed: Feb. 9, 2024
(86) PCT No.: PCT/JP2024/004546

§ 371 Date: Apr. 3, 2025

(30) Foreign Application Priority Data
Mar. 30, 2023 (JP) 2023-054856

(51) Int. Cl. G06F 30/20

(52) U.S. Cl. CPC G06F 30/20

(57) ABSTRACT

There is provided an apparatus including: a control parameter setting unit which sets a control parameter for a controller model which is included in a simulator of a cell culture system that controls an actuator by a controller according to a measurement result by a sensor, and which is a simulation model of the controller; a simulator execution unit which executes the simulator in a state in which the control parameter is set for the controller model; and a delay time setting unit which sets a delay time for a sensor model which is included in the simulator, and which is a simulation model of the sensor, in which after the delay time elapses from a time when a fluctuation occurs in a measurement target by the sensor model, the simulator execution unit causes the fluctuation to occur in a measurement result by the sensor model.

The contents of the following patent application(s) are incorporated herein by reference:
NO. 2023-054856 filed in JP on Mar. 30, 2023

### TECHNICAL FIELD

The present invention relates to an apparatus, a method, and a program.

### BACKGROUND ART

Patent Documents 1 to 9 and Non-Patent Document 1 disclose that “a method for the provision of optimized process specifications for a cell cultivation process in a reactor system from cultivation data of the cell cultivation process, comprising the steps of: acquiring cultivation data of the cell cultivation process; and adapting or generating at least one optimized process specification from acquired cultivation data by applying a Digital Twin obtainable according to the method of any one of claims 1 to 6”, or the like (claim 7 of Patent Document 4).

### Prior Art Documents

### Patent Documents

Patent Document 1: International Publication No. 2020252442
Patent Document 2: U.S. Patent Application Publication No. 2019-153381
Patent Document 3: International Publication No. WO2020-238918
Patent Document 4: Japanese Translation of PCT International Application Publication No. 2022-537799
Patent Document 5: Japanese Patent Application Publication No. 2019-041656
Patent Document 6: International Publication No. 2020-039683
Patent Document 7: International Publication No. 2021-166824
Patent Document 8: Japanese Patent Application Publication No. 2022-099096
Patent Document 9: Japanese Patent Application Publication No. 2018-049316

### Non-Patent Documents

Non-Patent Document 1: Sei Murakami, “Subcommittee 2, Current status and issues of continuous culture process,” Journal of the Society for Biotechnology, Japan, Vol. 97, No. 6, p. 338 to 341

### GENERAL DISCLOSURE

A first aspect of the present invention provides an apparatus including: a control parameter setting unit which sets a control parameter for a controller model which is included in a simulator of a cell culture system that controls an actuator by a controller according to a measurement result by a sensor, and which is a simulation model of the controller; and a simulator execution unit which executes the simulator in a state in which the control parameter is set for the controller model. The control parameter may indicate a control condition for the actuator, and may be a parameter of a feedback control.

The apparatus may further include a delay time setting unit which sets a delay time for a sensor model which is included in the simulator, and which is a simulation model of the sensor, and after the delay time elapses from a time when a fluctuation occurs in a measurement target by the sensor model, the simulator execution unit may cause the fluctuation to occur in a measurement result by the sensor model. The delay time may be a time between when the fluctuation occurs in the measurement target of the sensor model, and when the fluctuation occurs in the measurement result by the sensor model, and may be set based on culture data in the past.

In any of the apparatuses, at least one sensor in the cell culture system may be an in-line sensor which is immersed in a culture solution and measures a state of the culture solution, and the apparatus may further include a drift parameter setting unit which sets a parameter of a drift for an in-line sensor model which is included in the simulator, and which is a simulation model of the in-line sensor, and the simulator execution unit may cause the drift in accordance with the parameter of the drift, to occur for a measurement result of the in-line sensor model.

Any of the apparatuses may further include an uncertainty parameter setting unit which sets, for a culture tank model which is included in the simulator, and which is a simulation model of a culture tank of the cell culture system, one or more uncertainty parameters of a state of a cell in the culture tank model, and the simulator execution unit may cause an uncertainty in a state in accordance with the uncertainty parameter, to occur for the cell in the culture tank model.

The apparatuses may further include a determination unit which acquires, according to the simulator being executed multiple times in a state in which one of the one or more uncertainty parameters is set, a value indicating an execution result of each execution, to determine whether a distribution of the value satisfies a predetermined condition. The predetermined condition may be a condition in accordance with a control goal, and a value in accordance with a process value that is assumed by the sensor model, may be a target value (or in a reference range from the target value) thereof.

Any of the apparatuses may further include a determination unit which determines whether a value indicating an execution result satisfies a predetermined condition, according to the simulator being executed.

Any of the apparatuses having the determination unit may include an output unit which outputs the control parameter as a set value of the controller, according to the determination unit determining that the value indicating the execution result satisfies the predetermined condition.

A second aspect of the present invention provides a method including: setting a control parameter for a controller model which is included in a simulator of a cell culture system that controls an actuator by a controller according to a measurement result by a sensor, and which is a simulation model of the controller; and executing the simulator in a state in which the control parameter is set for the controller model.

A third aspect of the present invention provides a program which causes a computer to function as: a control parameter setting unit which sets a control parameter for a controller model which is included in a simulator of a cell culture system that controls an actuator by a controller according to a measurement result by a sensor, and which is a simulation model of the controller; and a simulator execution unit which executes the simulator in a state in which the control parameter is set for the controller model.

The summary clause does not necessarily describe all necessary features of the embodiments of the present invention. The present invention may also be a sub-combination of the features described above.

### BRIEF DESCRIPTION OF DRAWINGS

FIG. 1 shows a cell culture system 1 according to an embodiment.

FIG. 2 shows a control loop 15 of the cell culture system 1.

FIG. 3 shows a simulation apparatus 2 according to an embodiment.

FIG. 4 shows an operation of the simulation apparatus 2.

FIG. 5 shows an example of a computer 2200 in which a plurality of aspects of the present invention may be entirely or partially embodied.

### DESCRIPTION OF EMBODIMENTS

Hereinafter, the present invention will be described through embodiments of the invention, but the following embodiments do not limit the invention according to claims. In addition, not all of the combinations of features described in the embodiments are essential to the solution of the invention.

### 1. Cell Culture System 1

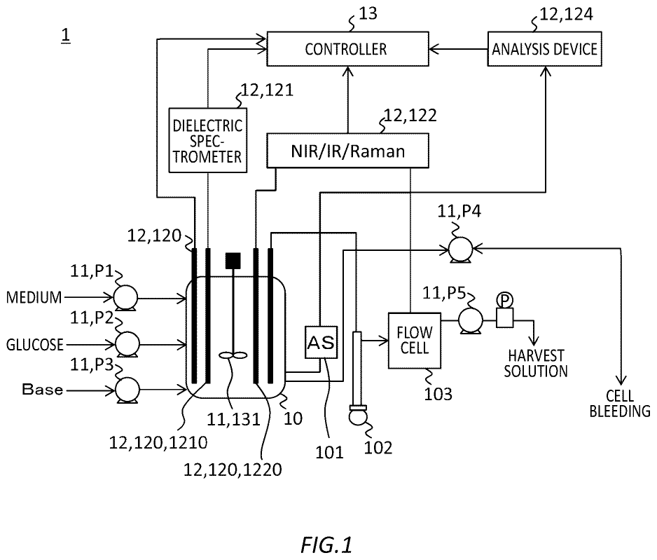

FIG. 1 shows the cell culture system 1 according to the present embodiment. The cell culture system 1 performs a culture to produce a target substance by a biological reaction. The cell culture system 1 may produce, for example, biopharmaceuticals such as antibody drugs, and may culture animal cells such as CHO cells. The cell culture system 1 may include a culture tank 10, one or more actuators 11, one or more sensors 12, and a controller 13. The culture tank 10, the one or more sensors 12, the controller 13, and the one or more actuators 11 may form one or more control loops (as an example, a closed control loop or an open control loop), and the cell culture system 1 may control the actuator 11 by the controller 13 according to a measurement result by the sensor 12.

### 1.1. Culture Tank 10

The culture tank 10 is a container for accommodating a medium and a cell to culture the cell. In order to cause cell proliferation to proceed, a temperature, a pH, an amount of dissolved oxygen (DO), and the like of a culture solution in the culture tank 10 may be appropriately managed by the actuator 11. As an example, a perfusion culture may be performed in the culture tank 10, and the medium may be continuously supplied to the culture tank 10 at a stage when the cell has proliferated to a certain extent (for example, on a second day to a third day from a start of the culture), for an amount of cell removal solution (a harvest solution) which is equal to the supplied amount, to be recovered from the culture tank 10. In this manner, a volume of the culture solution in the culture tank 10 may be kept to be constant.

It should be noted that the culture tank 10 may be provided with a manually operated sampling device 101 or a feed device (not shown). The sampling device 101 may sample the culture solution in the culture tank 10, and the feed device may administer a feed agent to the culture tank 10 for supplying a nutritional source. The sampling device 101 and the feed device may be the actuators 11 which are automatically operated.

### 1.2. Actuator 11

Each actuator 11 is a drive device which performs a physical movement, and may control at least one of a dissolved oxygen concentration, the pH, the temperature, a substrate concentration (a glucose concentration), a cell density, an amount of culture solution, a stirring speed, a pressure, a weight, a volume, a flow rate, or the like, in the culture solution. Each actuator may be any of a valve, a pump, a heater, a fan, a motor, and a switch.

In the present embodiment, as an example, the cell culture system 1 may have, as the actuator 11: a pump P1 which supplies the medium to the culture tank 10; a pump P2 which supplies glucose to the culture tank 10; a pump P3 which supplies an alkali (Base) to the culture tank 10; a stirring device 131 which stirs the culture solution in the culture tank 10; a pump P4 which discharges the cell together with the culture solution from the culture tank 10; and a pump P5 which discharges a harvest solution from the culture tank 10.

Among these actuators 11, the pump P5 may discharge the harvest solution in the culture tank 10 via a cell removal device 102 and a flow cell 103. The cell removal device 102 may have a filter action called a TFF (tangential flow filtration) or an ATF (alternating tangential flow filtration), and may discharge the harvest solution from the culture tank 10 while maintaining the cell in the culture tank 10.

### 1.3. Sensor 12

Each sensor 12 measures a state of the culture tank 10. Each sensor 12 may measure a process value (also referred to as a process parameter, PV) indicating the dissolved oxygen concentration, the pH, the temperature, the substrate concentration (the glucose concentration), the cell density, the amount of culture solution, the stirring speed, the pressure, the weight, the volume, the flow rate, or the like, in the culture solution.

In the present embodiment, as an example, the cell culture system 1 may have, as the sensor 12: a sensor (not shown) which measures the weight of the culture tank 10; a sensor (not shown) which measures the volume and the weight of a fluid that is supplied by the pumps P1 to P3; a sensor (not shown) which measures the density of cells; a dielectric spectrometer 121 which measures a dielectric constant of the culture solution; a near-infrared/infrared/Raman spectrometer 122 which measures nutritional and metabolic components in the culture solution; an analysis device 124 which analyzes a component of the culture solution; and an in-line sensor 120 which is immersed in the culture solution to measure a state of the culture solution.

Among these sensors 12, the dielectric spectrometer 121 may measure the dielectric constant of the culture solution according to a current flowing between electrodes 1210 inserted into the culture solution, and may further measure, from the dielectric constant that is measured, the number and a viability of the cells or the like in the culture solution.

The near-infrared/infrared/Raman spectrometer 122 may irradiate the culture solution with various types of measurement light (near-infrared light, infrared light, monochromatic light) via a light receiving and emitting sensor 1220 inserted into the culture solution, to measure a near-infrared spectrum, an infrared spectrum, a Raman spectrum, or the like of the light transmitted through or reflected from the culture solution; and may further measure, from the measured spectrum, glucose, lactate, ammonia, various types of amino acids, or the like in the culture solution. It should be noted that in the measurement by the near-infrared/infrared/Raman spectrometer 122, the measurement may be performed by: using a solution in which a component concentration has been adjusted in advance, to construct a calibration model; and converting the spectrum acquired from the light receiving and emitting sensor 1220 immersed in the culture tank 10, into the component concentration by the calibration model. The light receiving and emitting sensor 1220 may be provided in the flow cell 103.

The analysis device 124 may be, for example, a high performance liquid chromatography (HPLC) device, a cell culture analysis device, or the like. The analysis device 124 may analyze the component of the culture solution sampled from the culture tank 10 by the sampling device 101 at a reference interval (24 hours as an example), offline, that is, away from the culture tank 10.

The in-line sensor 120 may be immersed in the culture solution to measure the state of the culture solution during the execution of the cell culture. As an example, the in-line sensor 120 may measure at least one of fundamental physical and chemical parameters such as the pH, the amount of dissolved oxygen (DO), the temperature, a partial pressure of a gas (a partial pressure of oxygen, a partial pressure of carbon dioxide, or the like), an osmotic pressure, the nutritional component, the metabolic component, or a target product concentration, in the culture solution. The electrode 1210 of the dielectric spectrometer 121 and the light receiving and emitting sensor 1220 of the near-infrared/infrared/Raman spectrometer 122 described above may be the in-line sensors 120. It should be noted that a drift may occur in the measurement result by the in-line sensor 120 due to a change in a culture environment. An example of a factor that causes the change in the culture environment includes a fluctuation in a mechanical stress that occurs in the culture solution due to stirring or gas aeration, depletions of a nutrient and oxygen in the culture solution, an accumulation of a waste product (also referred to as debris, an impurity) such as lactate and ammonia that are produced by the cell.

Among the one or more sensors 12 included in the cell culture system 1, at least one sensor 12 may be provided with a noise removal filter which removes noise from the measurement result. The noise removal filter may cause a delay to occur between when the fluctuation occurs in a measurement target of the sensor 12, and when the fluctuation occurs in the measurement result by the sensor 12.

Each sensor 12 may perform the measurement in a cycle set in advance. Each sensor 12 may supply the process value as the measurement result to the controller 13. Each one of sensors 12 may perform a calibration at any timing.

It should be noted that the process value may be acquired by sampling the culture solution at a reference frequency (as an example, once a day), and performing the analysis by the analysis device 124. The cell density that is measured by the dielectric spectrometer 121 or the like may be calculated by analyzing an image captured by staining the culture solution. The nutritional/metabolic components that are measured by the near-infrared/infrared/Raman spectrometer 122 or the like may be measured by using an enzyme sensor or the like.

### 1.4. Controller 13

The controller 13 may control the one or more actuators 11 according to the measurement results by the one or more sensors 12. For example, the controller 13 may control the actuator 11 by supplying the actuator 11 with a control signal indicating a manipulated variable that is calculated according to the measurement result by the sensor 12. The controller 13 may scale (normalize, as an example) the measurement results of the one or more sensors 12 and then use the scaled measurement result to calculate the manipulated variable.

The controller 13 may control a culture process in the cell culture system 1 by controlling the one or more actuators 11 according to the measurement results by the one or more sensors 12. For example, the controller 13 may: monitor the state of the culture solution in the culture tank 10; execute various types of controls in relation to a perfusion culture process (operation controls of various types of pumps, motors, and the like, and a temperature control and the like); and control an amount of supply, an amount of recovery, an amount of aeration, a perfusion rate, or the like, in the culture tank 10. As an example, the controller 13 may add the nutritional component contained in the culture medium, or an enhancer that enhances the speed of the proliferation or the production of the cell, while controlling a fundamental process value such as the dissolved oxygen concentration, the pH, the temperature, the substrate concentration (the glucose concentration), the cell density, the amount of culture solution, and the stirring speed in the culture solution.

In the controller 13, a target value (also referred to as a set value, SV) of the process value may be set. When the target value is set, the controller 13 may calculate the manipulated variable for the process value to be the target value (or in a reference range from the target value), and control the actuator 11. When the cell culture system 1 has a plurality of control loops, the target value may be set in the controller 13 for each control loop. The target value of the process value may be set by an operator to achieve any control goal.

The control goal may be a goal in relation to a steady characteristic, and as an example, may be that the process value (PV) is in the reference range from the target value (SV) when the state of the culture tank 10 is a steady state. The control goal may be a goal in relation to a transient characteristic, and as an example may be that a settling time (also referred to as a response delay time) until the process value reaches the target value (or an inside of the reference range from the target value), is less than a reference time. The control goal may be a goal in relation to followability to the target value, and as an example, when the target value for any process value is changed and the cell culture is executed, the control goal may be that a rate (also referred to as a follow-up rate) at which the process value reaches the target value (or the inside of the reference range from the target value) exceeds a reference rate.

In the controller 13, a control parameter may be set. The control parameter may be a parameter indicating a control condition for the actuator 11, and may be, for example, a parameter of a feedback control (also referred to as a feedback control parameter). As an example, the feedback control parameter may be at least one of a proportional gain, an integral gain, or a derivative gain. The control parameter may be a parameter indicating an output frequency or a control cycle of the control signal to the actuator 11 (also referred to as a frequency parameter), and may be upper and lower limits of the manipulated variable provided to the actuator 11 for an instruction (also referred to as the manipulated variable for the actuator 11). The upper and lower limits of the manipulated variable may be set, as an example, at a rate of 0 (%) to 100 (%), −50 (%) to 50 (%), or the like.

The controller 13 may keep the cell density in the culture tank 10 in an appropriate range, by performing bleeding in which the cell is discharged from the culture tank 10 together with the culture solution by using the pump P4 according to the density of the cell reaching a reference value. The controller 13 may have a setting screen of the process parameter, a trend display function, a data storage/output function, or the like as a user interface.

### 2. Control Loop 15 of Cell Culture System 1

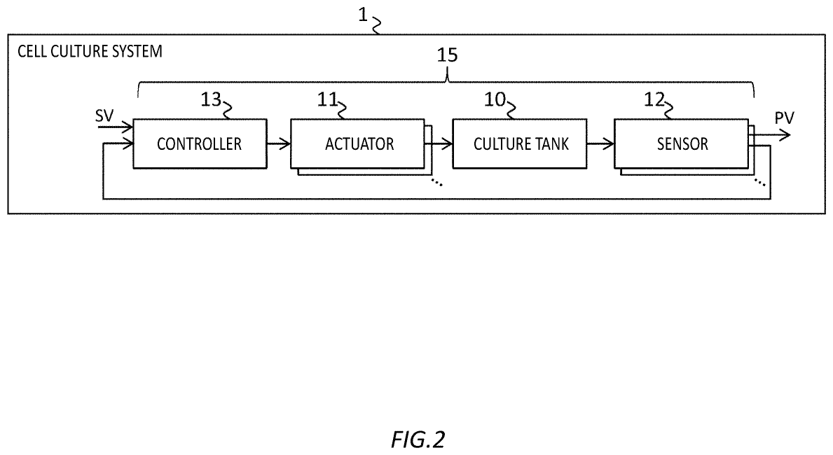

FIG. 2 shows the control loop 15 of the cell culture system 1. The controller 13, the actuator 11, the culture tank 10, and the sensor 12 of the cell culture system 1 may form the one or more closed control loops 15.

### 3. Simulation Apparatus 2

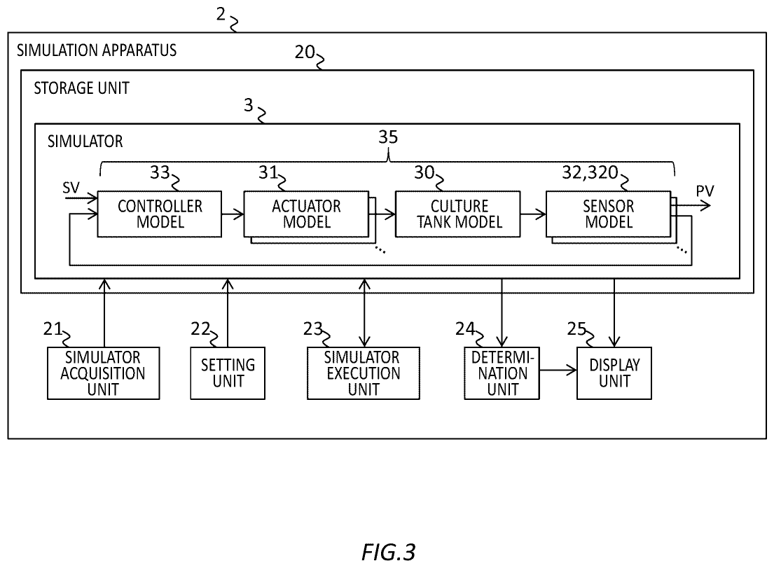

FIG. 3 shows the simulation apparatus 2 according to the present embodiment. The simulation apparatus 2 is an example of an apparatus, and performs a simulation of the cell culture system 1. The simulation apparatus 2 may have a storage unit 20, a simulator acquisition unit 21, a setting unit 22, a simulator execution unit 23, a determination unit 24, and a display unit 25.

### 3.1. Storage Unit 20

The storage unit 20 stores various types of information. The storage unit 20 according to the present embodiment may store a simulator 3 of the cell culture system 1.

### 3.1.1. Simulator 3

The simulator 3 may have a culture tank model 30 which is a simulation model of the culture tank 10; one or more actuator models 31 which are simulation models of the actuator 11; one or more sensor models 32 which are simulation models of the sensor 12; and a controller model 33 which is a simulation model of the controller 13. The controller model 33, the actuator model 31, the culture tank model 30, and the sensor model 32 may form one or more control loops 35 similar to that of the cell culture system 1. The simulation models may be a mathematical model, and may be respectively combined according to an input and output relationship of the control loop 35.

### 3.1.1(1). Culture Tank Model 30

The culture tank model 30 may reproduce a behavior of the culture tank 10. The behavior of the culture tank 10 may include a behavior of the culture solution or the like contained in the culture tank 10, and a behavior of the cell contained in the culture solution. The culture tank model 30 may be constructed to take account of a biological characteristic specific to the cell culture process (as an example, dynamics due to the cell). As an example, the culture tank model 30 may be a mechanistic model disclosed in References (1) and (2) which will be described below. For a control variable specific to a cell culture process, as long as it is possible to mathematically express the behavior during the process, the culture tank model 30 may be a data-driven model generated by machine learning, or a hybrid model (a model obtained by a combination of the mechanistic model and the data-driven model). The parameter indicating a dynamic characteristic of the culture tank model 30 may be set based on culture data or seed drain data in the past. In the culture tank model 30, an uncertainty parameter may be set. The uncertainty parameter may indicate an uncertainty of a state of the cell in the culture solution, and may be set as a random number component such as a normal random number for the culture tank model 30 disclosed in References (1) and (2). This makes it possible to perform the simulation taking account of the uncertainty of the cell state and even a variation between batches. When the culture tank model 30 is provided with the sampling device 101 and the feed device which are manually operated, these operating conditions may be defined for the culture tank model 30.

References (1): Sara Badr and five authors, “Integrated Design of Biopharmaceutical Manufacturing Processes: Operation Modes and Process Configurations for Monoclonal Antibody Production”, Computers and Chemical Engineering, Volume 153, 107422 (2021)

Reference (2): Martin Kornecki, Jochen Strube, “Process Analytical Technology for Advanced Process Control in Biologics Manufacturing with the Aid of Macroscopic Kinetic Modeling”, Bioengineering, 5, 2018

### 3.1.1(2). Actuator Model 31

Each actuator model 31 may reproduce the behavior of any actuator 11 which is a simulation target. Each actuator model 31 may be operated based on the manipulated variable that is supplied from the controller model 33. Among the one or more actuator models 31 included in the simulator 3, at least one actuator model 31 may perform unique processing on the manipulated variable (as an example, scaling or conversion processing) that is supplied, and may be operated based on the processed manipulated variable.

### 3.1.1(3). Sensor Model 32

Each sensor model 32 may reproduce the behavior of any sensor 12 which is the simulation target. Among the one or more sensor models 32 included in the simulator 3, at least one sensor model 32 may add pseudo noise to a value (also referred to as a true value) of the measurement target that is calculated by the culture tank model 30, and acquire a value to which the pseudo noise is added, as the process value. In the sensor model 32, the parameter of the pseudo noise that is added to the true value (as an example, an intensity, a shape, or the like of the noise) may be able to be set. The parameter of the noise may be set based on a specification of the sensor 12 which is the simulation target, or the culture data in the past. When reliability of the sensor 12 which is the simulation target is high, the parameter of the noise may be set for Gaussian white noise to be added.

Among the one or more sensor models 32 included in the simulator 3, at least one sensor model 32 may be provided with a simulation model (not shown) of the noise removal filter which removes the noise from the acquired process value. In the sensor model 32 provided with the noise removal filter, the parameter of the noise removal filter may be set. The parameter of the noise removal filter may be set based on the specification of the noise removal filter provided in the sensor 12 which is the simulation target, or the culture data in the past. The simulation model of the noise removal filter may cause the delay to occur between when the fluctuation occurs in the measurement target of the sensor model 32, and when the fluctuation occurs in the measurement result by the sensor model 32.

In at least one sensor model 32, among the one or more sensor models 32 included in the simulator 3, a delay time may be set between when the fluctuation occurs in the measurement target of the sensor model 32, and when the fluctuation occurs in the measurement result by the sensor model 32. The delay time may be set based on the culture data in the past, or the like.

Among the one or more sensor models 32 included in the simulator 3, at least one sensor model 32 may be an in-line sensor model 320 which is a simulation model of the in-line sensor 120. The in-line sensor model 320 may cause the drift to occur in the true value and acquire the drifted true value as the process value. In at least one in-line sensor model 320, the parameter of the drift (also referred to as a drift parameter) may be set. As an example, the parameter of the drift may indicate a magnitude of the drift that occurs according to the waste product in the culture solution increasing or decreasing by a unit amount; may indicate the magnitude of the drift that occurs per unit time; or may indicate the magnitude of the drift that occurs according to the temperature, the pressure, or the like increasing or decreasing by a unit amount. The parameter of the drift may be set by adjusting a coefficient of a functional expression for calculating the process value in the in-line sensor model 320. In the sensor model 32, a timing and a method of the calibration may be set.

In each sensor model 32, a measurement cycle may be set. The measurement cycle may be set based on the specification of the sensor 12 which is the simulation target.

### 3.1.1 (4). Controller Model 33

The controller model 33 may reproduce the behavior of the controller 13. The controller model 33 may control the one or more actuator models 31 according to the measurement results by the one or more sensor models 32. For example, the controller model 33 may control the actuator model 31 by supplying the actuator model 31 with the control signal indicating the manipulated variable that is calculated according to the measurement result by the sensor model 32. The controller model 33 may scale (normalize, as an example) the measurement results of the one or more sensors 12 and then use the scaled measurement result to calculate the manipulated variable.

In the controller model 33, the target value (SV) of the process value may be set, and the controller model 33 may calculate the manipulated variable for the process value to be the target value (or in the reference range from the target value), and control the actuator model 31. When the simulator 3 has the plurality of control loops 35, the target value may be set in the controller model 33 for each control loop 35.

In the controller model 33, the control parameter may be set. The control parameter may be a parameter indicating the control condition for the actuator model 31; for example, may be the feedback control parameter; may be the frequency parameter; or may be the upper and lower limits of the manipulated variable for the actuator 11.

In the controller model 33, the conditions for a start and an end of the control may be set. When influences of the control loops 35 on each other are taken into account or when influences of known disturbances are reduced, an amount of adjustment of the manipulated variable by feedforward may be set in the controller model 33.

### 3.2. Simulator Acquisition Unit 21

The simulator acquisition unit 21 acquires the simulator 3. The simulator acquisition unit 21 may acquire the simulator 3 generated by an external device, or may generate the simulator 3 according to an operation of the operator. The simulator 3 may be generated, as an example, in a development environment of a MATLAB (registered trademark)/Simulink, but may be generated in another development environment. The simulator acquisition unit 21 may cause the storage unit 20 to store the acquired simulator 3.

### 3.3. Setting Unit 22

The setting unit 22 may set various operating conditions for at least one of the controller model 33, the actuator model 31, the sensor model 32, or the culture tank model 30 of the simulator 3, according to the operation of the operator.

### 3.3.1. Set Content for Controller Model 33

The setting unit 22 may set the target value of the process value for the controller model 33. In a case of setting the target values for a plurality of process values that are measured by the plurality of sensor models 32, the setting unit 22 may set a common target value for each process value, or may set the target values different from each other for at least two process values. The setting unit 22 may appropriately set the target value while the simulator 3 is being executed, or may set the target value in advance before the simulator 3 is executed.

The setting unit 22 may be an example of a control parameter setting unit, and may set the control parameter for the controller model 33. The setting unit 22 may set the control parameter in advance before the simulator 3 is executed.

As an example, the setting unit 22 may set the feedback control parameter as the control parameter. When the plurality of control loops 35 are formed in the simulator 3, the setting unit 22 may set the feedback control parameter for at least one control loop 35. In a case of setting feedback control parameters for the plurality of control loops 35, the setting unit 22 may set a common feedback control parameter for each control loop 35, or may set the feedback control parameters different from each other for at least two control loops 35.

The setting unit 22 may set the frequency parameter as the control parameter. When the plurality of actuator models 31 are included in the simulator 3, the setting unit 22 may set the frequency parameter for at least one actuator model 31. In a case of setting frequency parameters for the plurality of actuator models 31, the setting unit 22 may set a common frequency parameter for each actuator model 31, or may set the frequency parameters different from each other for at least two actuator models 31.

The setting unit 22 may set the upper and lower limits of the manipulated variable for the actuator model 31 as the control parameter. When the plurality of actuator models 31 are included in the simulator 3, the setting unit 22 may set the upper and lower limits of the manipulated variable for at least one actuator model 31. In a case of setting the upper and lower limits of the manipulated variables for the plurality of actuator models 31, the setting unit 22 may set common upper and lower limits of the manipulated variable for each actuator model 31, or may set the upper and lower limits of the manipulated variables different from each other for at least two actuator models 31.

The setting unit 22 may set the amount of adjustment of the manipulated variable by the feedforward. In a case of setting the amounts of adjustment for the plurality of actuator models 31, the setting unit 22 may set a common amount of adjustment for each actuator model 31, or may set the amounts of adjustment different from each other for at least two actuator models 31. The setting unit 22 may set the amount of adjustment in advance before the simulator 3 is executed.

### 3.3.2. Set Content for Sensor Model 32

The setting unit 22 may set the noise parameter for at least one sensor model 32. In a case of setting noise parameters for the plurality of sensor models 32, the setting unit 22 may set a common parameter for each sensor model 32, or may set the parameters different from each other for at least two sensor models 32. The setting unit 22 may set the parameter of the noise in advance before the simulator 3 is executed.

The setting unit 22 may set the parameter of the noise removal filter for at least one sensor model 32. In a case of setting the parameters of the noise removal filters for the plurality of sensor models 32, the setting unit 22 may set a common parameter for each sensor model 32, or may set the parameters different from each other for at least two sensor models 32. The setting unit 22 may set the parameter of the noise removal filter in advance before the simulator 3 is executed.

The setting unit 22 may be an example of a delay time setting unit, and may set the delay time for at least one sensor model 32. In a case of setting delay times for the plurality of sensor models 32, the setting unit 22 may set a common delay time for each sensor model 32, or may set the delay times different from each other for at least two sensor models 32. The setting unit 22 may set the delay time in advance before the simulator 3 is executed.

The setting unit 22 may be an example of a drift parameter setting unit, and may set the drift parameter for at least one in-line sensor model 320. In a case of setting drift parameters for a plurality of in-line sensor models 320, the setting unit 22 may set a common drift parameter for each in-line sensor model 320, or may set the drift parameters different from each other for at least two in-line sensor models 320. The setting unit 22 may set the drift parameter in advance before the simulator 3 is executed.

### 3.3.3. Set Content for Culture Tank Model 30

The setting unit 22 may be an example of an uncertainty parameter setting unit, and may set, for the culture tank model 30, the uncertainty parameter of the state of the cell in the culture tank model 30. The setting unit 22 may set, together with the uncertainty parameter, the number of times of execution of the simulation to which the uncertainty parameter is applied. The setting unit 22 may set these contents in advance before the simulator 3 is executed.

### 3.3.4. Other Set Content

The setting unit 22 may set the control goal for the simulation by the operation of the operator.

The control goal may be, for example, a goal in relation to a steady characteristic, and as an example, may be that the process value (PV) is in the reference range from the target value (SV) when the state of the culture tank model 30 is a steady state. In this case, the setting unit 22 may set the reference range for the process value to be kept in when the state of the culture tank model 30 is the steady state.

The control goal may be a goal in relation to a transient characteristic, and as an example may be that the settling time (also referred to as the response delay time) until the process value reaches the target value (or the inside of the reference range from the target value) is less than a reference time. The control goal may be a goal in relation to followability to the target value, and as an example, when the target value for any process value is changed and the cell culture is executed, the control goal may be that the follow-up rate at which the process value reaches the target value (or the inside of the reference range from the target value) exceeds the reference rate. In these cases, the setting unit 22 may set the reference time for the settling time to fall below, and may set the reference rate for the follow-up rate to exceed.

The setting unit 22 may set the environment of the simulator 3 by the operation of the operator. As an example, the setting unit 22 may set a type of solver that is used by the simulator 3 (for example, an Euler method, Runge-Kutta methods, or the like), or may set global variables (as an example, the simulation section (that is, a culture period)) that are used by the plurality of simulation models.

The setting unit 22 may supply the set content to the simulator 3, or may supply the set content to the simulator execution unit 23. The setting unit 22 may supply the set content of the control goal to the determination unit 24.

### 3.4. Simulator Execution Unit 23

The simulator execution unit 23 executes the simulator 3.

The simulator execution unit 23 may execute the simulator 3 in a state in which the target value (SV) of the process value is set for the controller model 33. The simulator execution unit 23 may cause the controller model 33 to drive the actuator model 31 such that the process value which is measured by the sensor model 32 approaches the target value.

The simulator execution unit 23 may execute the simulator 3 in a state in which the control parameter is set for the controller model 33. The simulator execution unit 23 may cause the controller model 33 to perform the feedback control in accordance with the set feedback control parameter. The simulator execution unit 23 may cause the control signal to be output from the controller model 33 to the actuator model 31 at the output frequency or the control cycle in accordance with the set frequency parameter. The simulator execution unit 23 may cause the control signal indicating the manipulated variable in accordance with the set upper and lower limits of the manipulated variable, to be output from the controller model 33 to the actuator model 31.

The simulator execution unit 23 may execute the simulator 3 in a state in which the amount of adjustment of the feedforward is set for the controller model 33. The simulator execution unit 23 may cause the control signal indicating the manipulated variable in accordance with the set content, to be output from the controller model 33 to the actuator model 31.

The simulator execution unit 23 may execute the simulator 3 in a state in which the uncertainty parameter is set for the culture tank model 30. The simulator execution unit 23 may cause the uncertainty in a state in accordance with the uncertainty parameter, to occur for the cell in the culture tank model 30. The simulator execution unit 23 may execute the simulator 3 the number of times of execution predetermined by the setting unit 22, according to the uncertainty parameter being set.

The simulator execution unit 23 may execute the simulator 3 in a state in which the parameter of the noise is set for the sensor model 32. The simulator execution unit 23 may cause the sensor model 32 to acquire the process value obtained by adding the noise to the true value of the measurement target that is calculated by the culture tank model 30, according to the parameter of the noise.

The simulator execution unit 23 may execute the simulator 3 in a state in which the parameter of the noise removal filter is set for the sensor model 32. The simulator execution unit 23 may cause the sensor model 32 to acquire the process value obtained by removing the noise according to the parameter of the noise removal filter.

The simulator execution unit 23 may execute the simulator 3 in a state in which the delay time is set for the sensor model 32. After the delay time elapses from a time when the fluctuation occurs in the measurement target by the sensor model 32, the simulator execution unit 23 may cause the fluctuation to occur in the measurement result by the sensor model 32.

The simulator execution unit 23 may execute the simulator 3 in a state in which the drift parameter is set for the in-line sensor model 320. The simulator execution unit 23 may cause the drift in accordance with the drift parameter to occur for a measurement result of the in-line sensor model 320.

The simulator execution unit 23 may execute the simulator 3 in a state in which the measurement cycle is set for the sensor model 32. The simulator execution unit 23 may cause each sensor model 32 to acquire the process value at the measurement cycle.

The simulator execution unit 23 may execute the simulator 3 according to the setting of the environment that is set by the setting unit 22. As an example, when the type of the solver is set, the simulator execution unit 23 may perform the calculations by using the set solver. When the simulation section is set, the simulation may be performed in the set simulation section.

The simulator execution unit 23 may cause the storage unit 20 to store an execution result of the simulation by the simulator 3. The execution result of the simulation may include time-series data obtained by each simulation model, and the set content for the simulation model.

### 3.5. Determination Unit 24

The determination unit 24 determines whether a value indicating the execution result satisfies a predetermined condition, according to the simulator 3 being executed. The predetermined condition may be a condition in accordance with the control goal, and is also referred to as a goal condition. The determination unit 24 may acquire the value indicating the execution result from the simulator 3.

The values indicating the execution results may be values in accordance with the process values (as an example, the process value itself) that are measured by the one or more sensor models 32 when a state of the culture tank 10 is in a steady state. In this case, the goal condition may be that the process value is the target value (or in the reference range from the target value) thereof.

The value indicating the execution result may be the settling time until the process value reaches the target value (or the inside of the reference range from the target value). In this case, the goal condition may be that the settling time is less than the reference time, may be that the settling time is less than a settling time when the simulator 3 was previously executed, or may be that the settling time is less than a settling time in a case of the cell culture performed in the cell culture system 1.

The value indicating the execution result may be the follow-up rate at which the process value reaches the target value (or the inside of the reference range from the target value) when the target value for any process value is changed and the simulator 3 is executed. In this case, the goal condition may be that the follow-up rate exceeds the reference rate thereof, may be that the follow-up rate exceeds a follow-up rate when the simulator 3 was previously executed, or may be that the follow-up rate exceeds a follow-up rate in a case of the cell culture performed in the cell culture system 1.

The determination unit 24 may acquire, according to the simulator 3 being executed multiple times in a state in which one uncertainty parameter is set by the setting unit 22, the value indicating the execution result of each execution, to determine whether a distribution of the value satisfies a predetermined condition (also referred to as a distribution condition). The distribution condition may be that the value indicating the execution result is kept in the reference range set in advance, and in addition or instead of this, and may be that a variance or a standard deviation of the value indicating the execution result is kept in the reference range set in advance. The determination unit 24 may supply a determination result to the display unit 25.

### 3.6. Display Unit 25

The display unit 25 displays various types of information. The display unit 25 may display the value indicating the execution result of the simulator 3, and the determination result by the determination unit 24, in a combination. The display unit 25 may be an example of an output unit, and may display the control parameter set by the setting unit 22 as the set value of the controller 13, according to the determination unit 24 determining that the value indicating the execution result of the simulator 3 satisfies the goal condition.

With the above simulation apparatus, the simulator 3 is executed in the state in which the control parameter is set for the controller model 33 included in the simulator 3 of the cell culture system 1. Accordingly, by adjusting the control parameter and executing the simulator 3, it is possible to specify a suitable control parameter for realizing any goal and to apply the specified suitable control parameter to the controller 13 of the cell culture system 1. In addition, in comparison with a case where the control parameter is changed in the cell culture system 1 and the cell culture is repeated, it is possible to reduce a time, a raw material, labor, or the like required for specifying the suitable control parameter.

In addition, after the delay time elapses from a time when the delay time is set for the sensor model 32 which is the simulation model of the sensor 12 and the fluctuation occurs in the measurement target by the sensor model 32, the fluctuation in the measurement result by the sensor model 32 occurs. Accordingly, it is possible to execute the simulator 3 in a state in which the delay time of the sensor model 32 is adapted for the actual sensor 12.

In addition, the drift parameter is set for the in-line sensor model 320 which is the simulation model of the in-line sensor 120, and the drift in accordance with the drift parameter occurs for the measurement result of the in-line sensor model 320. Accordingly, it is possible to execute the simulator 3 in a state in which the drift due to the waste product or the like occurring along with the culture proceeding is adapted for the real environment.

In addition, the uncertainty parameter of the state of the cell is set for the culture tank model 30 which is the simulation model of the culture tank, and the uncertainty in a state in accordance with the uncertainty parameter, occurs. Accordingly, it is possible to execute the simulator 3 in a state in which the state of the cell in the culture tank model 30 is adapted for the uncertainty of the cell state in the real environment.

In addition, according to the simulator 3 being executed, it is determined whether the value indicating the execution result satisfies a predetermined goal condition. Accordingly, when the cell culture is performed in the real environment, it is possible to check in advance whether a culture result satisfies the goal condition.

In addition, according to the determination that the value indicating the execution result of the simulator 3 satisfies the goal condition, the control parameter is output as the set value of the controller 13, and thus it is possible to apply the control parameter for satisfying the goal condition to the controller 13 of the cell culture system 1 and perform the cell culture in the real environment.

In addition, according to the simulator 3 being executed multiple times in a state in which one uncertainty parameter is set, the value indicating the execution result of each execution is acquired, and it is determined whether the distribution of the value satisfies a predetermined distribution condition. Accordingly, when the cell culture is performed multiple times in the real environment, it is possible to check in advance whether a plurality of culture results satisfy the distribution condition as a whole.

In addition, the simulation models included in the simulator 3 are combined with each other according to an input and output relationship, and thus by grasping the influences between the simulation models, it is possible to grasp the influences between the components (in the present embodiment, as an example, the controller 13, the actuator 11, the culture tank 10, and the sensor 12) of the cell culture system 1 which is the simulation target. In addition, for each simulation model, it is possible to grasp a degree of the influence on a control performance of the control loop 35, and to use the grasped influence for setting the control parameter, and thus it is possible to easily set the control parameter.

### 4. Operation

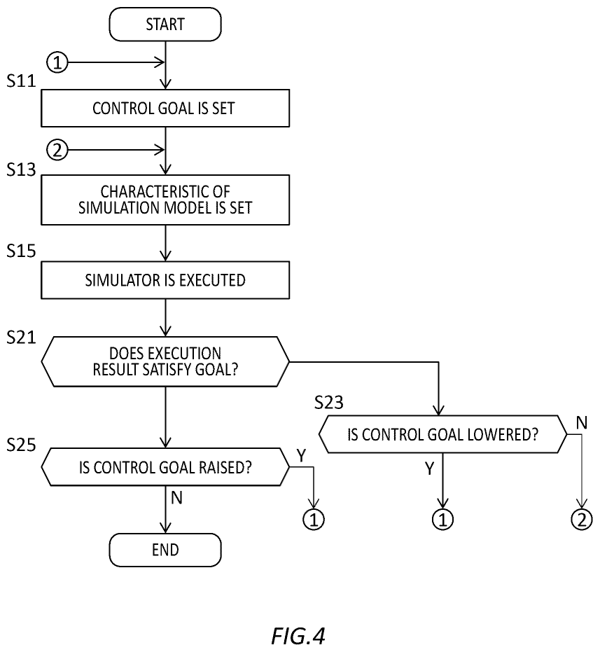

FIG. 4 shows an operation of the simulation apparatus 2. The simulation apparatus 2 simulates the cell culture system 1 by performing processing of steps S11 to S25, and determines the control parameter for achieving a desired control goal. It should be noted that in the present embodiment, the description will be made on an assumption that the simulator 3 is acquired in advance by the simulator acquisition unit 21 in the simulation apparatus 2, as an example.

In step S11, the setting unit 22 sets the control goal for the simulation according to the operation of the operator. The setting unit 22 may set one or more control goals. In this manner, the goal condition may be set in accordance with the control goal. The setting unit 22 may further set a distribution goal indicating the distribution condition of the value indicating the execution result of the simulator 3, according to the operation of the operator. In this manner, the distribution condition may be set in accordance with the distribution goal. The setting unit 22 may further set the environment of the simulator according to the operation of the operator.

In step S13, the setting unit 22 sets the characteristic of each simulation model according to the operation of the operator. The setting unit 22 may set one or more control parameters (as an example, the feedback control parameter, the frequency parameter, or the upper and lower limits of the manipulated variable) for the controller model 33; may set the delay time for the sensor model 32; may set the drift parameter for the in-line sensor model 320; and may set the uncertainty parameter for the culture tank model 30.

In step S15, the simulator execution unit 23 executes the simulator 3. The simulator execution unit 23 may execute the simulator 3 in a state in which the set content is set by steps S11 and S13. As an example, the simulator execution unit 23 may execute the simulator 3 in the state in which the control parameter is set for the controller model 33. When the uncertainty parameter is set in step S13, the simulator execution unit 23 may execute the simulator 3 multiple times in the state in which the uncertainty parameter is set.

In step S21, the determination unit 24 determines whether the value indicating the execution result of step S15 satisfies the goal condition.

When the simulator 3 is executed multiple times in a state in which one uncertainty parameter is set in step S15, the determination unit 24 may acquire the value indicating the execution result of each execution, and further determine whether the distribution of the value satisfies the distribution condition. The determination unit 24 may cause the display unit 25 to display the determination result together with the execution result of the simulator 3, in a combination.

If it is determined in step S21 that the control condition is not satisfied (step S21; No), the processing may proceed to step S23. If it is determined in step S21 that the control condition is satisfied (step S21; Yes), the processing may proceed to step S25.

In step S23, the determination unit 24 determines whether an operation to an effect that the control goal is lowered, is performed by the operator. If it is determined that the operation to the effect that the control goal is lowered, is not performed (step S23; No), the processing may proceed to step S13 described above. In this manner, the characteristic of each simulator model is reset, and the simulator 3 is executed. If it is determined that the operation to the effect that the control goal is lowered, is performed (step S23; Yes), the processing may proceed to step S11 described above. In this manner, the control goal is reset to be low such that the control goal is able to be realized, and the simulator 3 is executed.

In step S25, the determination unit 24 determines whether an operation to an effect that the control goal is raised, is performed by the operator. If it is determined that the operation to the effect that the control goal is raised, is performed (step S25; Yes), the processing may proceed to step S11 described above. In this manner, the control goal is reset to be high such that the control goal is stringent, and the simulator 3 is executed. If it is determined that the operation to the effect that the control goal is raised, is not performed (step S25; No), the operation may end. In this manner, the control parameter and the characteristic of the simulation model by which it is possible to achieve the desired control goal, are specified. It should be noted that when the characteristic of the simulation model or the like is changed and the simulation is performed, the present operation may be performed again from the beginning.

### 5. Modification Example

It should be noted that in the above embodiment, the simulation apparatus 2 has been described to have the simulator acquisition unit 21, the determination unit 24, and the display unit 25; however, the simulation apparatus 2 may not have any of these.

In addition, the display unit 25 has been described as an example of the output unit; however, another configuration may be used as the output unit as long as the control parameter is output as the set value of the controller. For example, the output unit may be a control device of the controller 13; and may output the set control parameter to the controller 13 as the set value, according to the determination unit 24 determining that the value indicating the execution result of the simulator 3 satisfies the goal condition, to cause the control in accordance with the control parameter to be performed. In this case, the simulator 3 may be used as a digital twin of the cell culture system 1, and may predict the result of cell culture in the cell culture system 1 by real data, such as the process value indicating the state of the cell culture system 1, being input, and the simulation being performed. When the prediction result is not good, the control parameter of the controller 13 in the cell culture system 1 may be adjusted, or the sensor 12 may be calibrated.

In addition, the controller 13 has been described to determine the manipulated variable by the feedback control; however, in addition to or instead of this, the manipulated variable may be determined by a model predictive control in which a prediction model (as an example, the culture tank model 30 of the simulator 3) that predicts the behavior of the culture tank 10, is used. For example, the controller 13 may determine the manipulated variable to optimize a reward value that is determined by a reward function set in advance. The reward function may be a function that has one or more measurement values as variables, or may be a function that has a settling time as a variable. When the controller 13 performs the model predictive control, the setting unit 22 may set, as the control parameters for the controller model 33, at least one of a weight coefficient of the reward function, a prediction section (Prediction Horizon) for acquiring the output from the prediction model, or a control section (Control Horizon) for controlling the input to the prediction model. In this case, the simulator execution unit 23 may execute the simulator 3 in a state in which these control parameters are set for the controller model 33. For example, the simulator execution unit 23 may cause the controller model 33 to perform the model predictive control, to determine the manipulated variable for optimizing the reward value that is determined by the reward function in which the weight coefficient is set. The simulator execution unit 23 may cause the controller model 33 to perform the model predictive control in accordance with the set prediction interval and control interval.

In addition, the cell culture system 1 has been described to perform the perfusion culture; however, the culture may be performed by another method such as a fed-batch culture or a continuous culture.

In addition, the cell culture system 1 has been described to culture an animal cell such as a CHO cell; however, a vertebrate cell other than the CHO cell may be cultured, a cell of a shellfish, an insect, or the like may be cultured, and a human cell, a plant cell, a microbial cell may be cultured.

In addition, various embodiments of the present invention may be described with reference to flowcharts and block diagrams, wherein the block may serve as (1) a stage in a process in which an operation is performed, or (2) a section of an apparatus having a role of performing an operation. Certain stages and sections may be implemented by a dedicated circuit, a programmable circuit supplied with computer-readable instructions stored on computer-readable media, and/or processors supplied with computer-readable instructions stored on computer-readable media. The dedicated circuit may include digital and/or analog hardware circuits, and may include integrated circuits (IC) and/or discrete circuits. The programmable circuit may include a reconfigurable hardware circuit including logical AND, logical OR, logical XOR, logical NAND, logical NOR, and other logical operations, a memory element such as a flip-flop, a register, a field programmable gate array (FPGA) and a programmable logic array (PLA), and the like.

A computer-readable medium may include any tangible device that can store instructions to be executed by a suitable device, and as a result, the computer-readable medium having instructions stored thereon includes an article of manufacture including instructions which can be executed in order to create means for performing operations designated in the flowcharts or the block diagrams. Examples of the computer-readable medium may include an electronic storage medium, a magnetic storage medium, an optical storage medium, an electromagnetic storage medium, a semiconductor storage medium, and the like. More specific examples of the computer-readable medium may include a floppy (registered trademark) disk, a diskette, a hard disk, a random access memory (RAM), a read-only memory (ROM), an erasable programmable read-only memory (EPROM or flash memory), an electrically erasable programmable read-only memory (EEPROM), a static random access memory (SRAM), a compact disc read-only memory (CD-ROM), a digital versatile disc (DVD), a Blu-ray (registered trademark) disc, a memory stick, an integrated circuit card, and the like.

The computer-readable instruction may include: an assembler instruction, an instruction-set-architecture (ISA) instruction; a machine instruction; a machine dependent instruction; a microcode; a firmware instruction; state-setting data; or either a source code or an object code written in any combination of one or more programming languages, including an object oriented programming language such as Smalltalk (registered trademark), JAVA (registered trademark), C++, or the like, and a conventional procedural programming language such as a “C” programming language or a similar programming language.

Computer-readable instructions may be provided to a processor of a general-purpose computer, special purpose computer, or other programmable data processing apparatuses, or to the programmable circuit, locally or via a local area network (LAN), a wide area network (WAN) such as the Internet, or the like, to execute the computer-readable instructions to create means for performing operations specified in the flowcharts or the block diagrams. Examples of the processor include a computer processor, a processing unit, a microprocessor, a digital signal processor, a controller, a microcontroller, and the like.

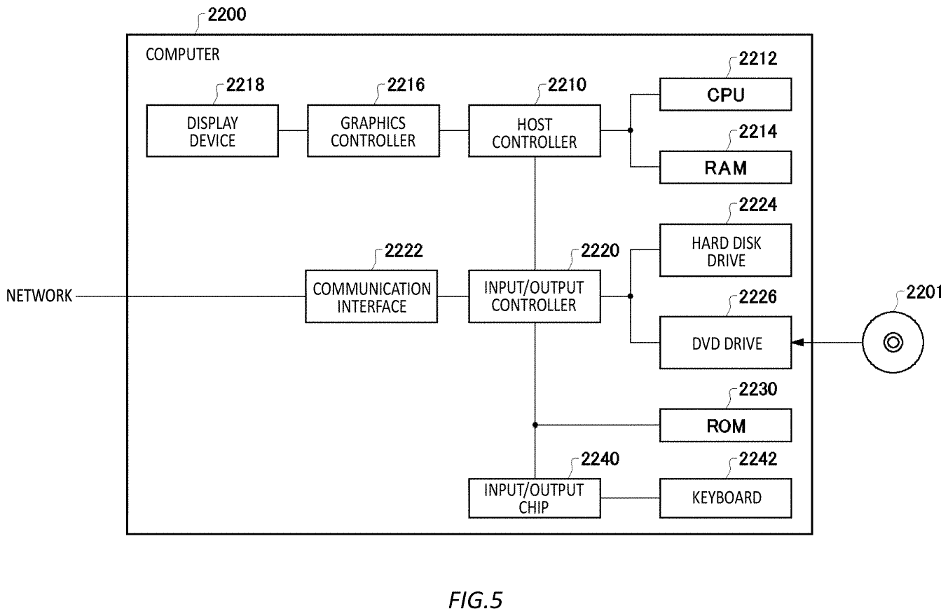

FIG. 5 shows an example of a computer 2200 in which a plurality of aspects of the present invention may be entirely or partially embodied. A program installed in the computer 2200 may cause the computer 2200 to function as an operation associated with the apparatus according to the embodiments of the present invention or as one or more sections of the apparatuses, or may cause the operation or the one or more sections to be executed, and/or may cause the computer 2200 to execute a process according to the embodiments of the present invention or a stage of the process. Such programs may be executed by a CPU 2212 in order to cause the computer 2200 to perform certain operations associated with some or all of the blocks in the flowcharts and the block diagrams described in the present specification.

The computer 2200 according to the present embodiment includes the CPU 2212, a RAM 2214, a graphics controller 2216, and a display device 2218, which are mutually connected by a host controller 2210. The computer 2200 further includes input/output units such as a communication interface 2222, a hard disk drive 2224, a DVD-ROM drive 2226, and an IC card drive, which are connected to the host controller 2210 via an input/output controller 2220. The computer also includes legacy input/output units such as a ROM 2230 and a keyboard 2242, which are connected to the input/output controller 2220 via an input/output chip 2240.

The CPU 2212 operates according to programs stored in the ROM 2230 and the RAM 2214, thereby controlling each unit. The graphics controller 2216 obtains image data generated by the CPU 2212 on a frame buffer or the like provided in the RAM 2214 or in itself, and causes the image data to be displayed on the display device 2218.

The communication interface 2222 communicates with other electronic devices via a network. The hard disk drive 2224 stores programs and data used by the CPU 2212 in the computer 2200. The DVD-ROM drive 2226 reads the programs or the data from the DVD-ROM 2201, and provides the hard disk drive 2224 with the programs or the data via the RAM 2214. The IC card drive reads the programs and the data from the IC card, and/or writes the programs and the data to the IC card.

The ROM 2230 stores therein boot programs and the like executed by the computer 2200 at the time of activation, and/or programs that depend on the hardware of the computer 2200. The input/output chip 2240 may also connect various input/output units to the input/output controller 2220 via a parallel port, a serial port, a keyboard port, a mouse port, and the like.

The program is provided by a computer-readable medium such as the DVD-ROM 2201 or the IC card. The program is read from a computer-readable medium, installed in the hard disk drive 2224, the RAM 2214, or the ROM 2230 which are also examples of the computer-readable medium, and executed by the CPU 2212. The information processing written in these programs is read by the computer 2200 and provides cooperation between the programs and the above-described various types of hardware resources. The apparatus or method may be constituted by realizing operations or processing of information according to use of the computer 2200.

For example, in a case where communication is performed between the computer 2200 and an external device, the CPU 2212 may execute a communication program loaded in the RAM 2214 and instruct the communication interface 2222 to perform communication processing on the basis of a process written in the communication program. Under the control of the CPU 2212, the communication interface 2222 reads transmission data stored in a transmission buffer processing area provided in a recording medium such as the RAM 2214, the hard disk drive 2224, the DVD-ROM 2201, or the IC card, transmits the read transmission data to the network, or writes reception data received from the network in a reception buffer processing area or the like provided on the recording medium.

In addition, the CPU 2212 may cause the RAM 2214 to read all or a necessary part of a file or database stored in an external recording medium such as the hard disk drive 2224, the DVD-ROM drive 2226 (DVD-ROM 2201), the IC card, or the like, and may execute various types of processing on data on the RAM 2214. Next, the CPU 2212 writes back the processed data to the external recording medium.

Various types of information such as various types of programs, data, tables, and databases may be stored in a recording medium and subjected to information processing. The CPU 2212 may execute various types of processing on the data read from the RAM 2214 to write back a result to the RAM 2214, the processing being described throughout the present disclosure, designated by instruction sequences of the programs, and including various types of operations, information processing, condition determinations, conditional branching, unconditional branching, information searches/replacements, or the like. In addition, the CPU 2212 may search for information in a file, a database, etc., in the recording medium. For example, when a plurality of entries, each having an attribute value of a first attribute associated with an attribute value of a second attribute, are stored in the recording medium, the CPU 2212 may search for an entry matching the condition whose attribute value of the first attribute is designated, from among the plurality of entries, and read the attribute value of the second attribute stored in the entry, thereby obtaining the attribute value of the second attribute associated with the first attribute satisfying the predetermined condition.

The programs or software modules described above may be stored in a computer-readable medium on or near the computer 2200. In addition, a recording medium such as a hard disk or a RAM provided in a server system connected to a dedicated communication network or the Internet can be used as a computer-readable medium, thereby providing a program to the computer 2200 via the network.

While the present invention has been described by way of the embodiments, the technical scope of the present invention is not limited to the above-described embodiments. It is apparent to persons skilled in the art that various alterations or improvements can be made to the above-described embodiments. It is also apparent from the description of the claims that embodiments added with such alterations or improvements can be included in the technical scope of the present invention.

Note that the operations, procedures, steps, and stages of each process performed by an apparatus, system, program, and method shown in the claims, embodiments, or diagrams can be performed in any order as long as the order is not indicated by “prior to,” “before,” or the like and as long as the output from a previous process is not used in a later process. Even if the operation flow is described by using phrases such as “first” or “next” in the scope of the claims, specification, or drawings, it does not necessarily mean that the process must be performed in this order.

### REFERENCE SIGNS LIST

1: cell culture system;
2: simulation apparatus;
3: simulator;
10: culture tank;
11: actuator;
13: controller;
15: control loop;
20: storage unit;
21: simulator acquisition unit;
22: setting unit;
23: simulator execution unit;
24: determination unit;
25: display unit;
30: culture tank model;
31: actuator model;
32: sensor model;
33: controller model;
35: control loop;
101: sampling device;
102: cell removal device;
103: flow cell;
120: in-line sensor;
121: dielectric spectrometer;
122: near-infrared/infrared/Raman spectrometer;
124: analysis device;
131: stirring device;
1210: electrode;
1220: light receiving and emitting sensor;
2200: computer;
2201: DVD-ROM;
2210: host controller;
2212: CPU;
2214: RAM;
2216: graphics controller;
2218: display device;
2220: input/output controller;
2222: communication interface;
2224: hard disk drive;
2226: DVD-ROM drive;
2230: ROM;
2240: input/output chip;
2242: keyboard.

1. An apparatus comprising:
a control parameter setting unit which sets a control parameter for a controller model which is included in a simulator of a cell culture system that controls an actuator by a controller according to a measurement result by a sensor, and which is a simulation model of the controller; and
a simulator execution unit which executes the simulator in a state in which the control parameter is set for the controller model.

2. The apparatus according to claim 1, further comprising:
a delay time setting unit which sets a delay time for a sensor model which is included in the simulator, and which is a simulation model of the sensor, wherein
after the delay time elapses from a time when a fluctuation occurs in a measurement target by the sensor model, the simulator execution unit causes the fluctuation to occur in a measurement result by the sensor model.

3. The apparatus according to claim 1, wherein
at least one sensor in the cell culture system is an in-line sensor which is immersed in a culture solution and measures a state of the culture solution,
the apparatus further comprising a drift parameter setting unit which sets a parameter of a drift for an in-line sensor model which is included in the simulator, and which is a simulation model of the in-line sensor; wherein
the simulator execution unit causes the drift in accordance with the parameter of the drift, to occur for a measurement result of the in-line sensor model.

4. The apparatus according to claim 1, further comprising:
an uncertainty parameter setting unit which sets, for a culture tank model which is included in the simulator, and which is a simulation model of a culture tank of the cell culture system, one or more uncertainty parameters of a state of a cell in the culture tank model, wherein
the simulator execution unit causes an uncertainty in a state in accordance with the uncertainty parameter, to occur for the cell in the culture tank model.

5. The apparatus according to claim 4, further comprising: a determination unit which acquires, according to the simulator being executed multiple times in a state in which one of the one or more uncertainty parameters is set, a value indicating an execution result of each execution, to determine whether a distribution of the value satisfies a predetermined condition.

6. The apparatus according to claim 1, further comprising: a determination unit which determines whether a value indicating an execution result satisfies a predetermined condition, according to the simulator being executed.

7. The apparatus according to claim 6, further comprising: an output unit which outputs the control parameter as a set value of the controller, according to the determination unit determining that the value indicating the execution result satisfies the predetermined condition.

8. A method comprising:
setting a control parameter for a controller model which is included in a simulator of a cell culture system that controls an actuator by a controller according to a measurement result by a sensor, and which is a simulation model of the controller; and
executing the simulator in a state in which the control parameter is set for the controller model.

9. A non-transitory computer readable medium having recorded thereon a program which causes a computer to function as:
a control parameter setting unit which sets a control parameter for a controller model which is included in a simulator of a cell culture system that controls an actuator by a controller according to a measurement result by a sensor, and which is a simulation model of the controller; and
a simulator execution unit which executes the simulator in a state in which the control parameter is set for the controller model.

10. The apparatus according to claim 2, wherein
at least one sensor in the cell culture system is an in-line sensor which is immersed in a culture solution and measures a state of the culture solution,
the apparatus further comprising a drift parameter setting unit which sets a parameter of a drift for an in-line sensor model which is included in the simulator, and which is a simulation model of the in-line sensor; wherein
the simulator execution unit causes the drift in accordance with the parameter of the drift, to occur for a measurement result of the in-line sensor model.

11. The apparatus according to claim 2, further comprising:
an uncertainty parameter setting unit which sets, for a culture tank model which is included in the simulator, and which is a simulation model of a culture tank of the cell culture system, one or more uncertainty parameters of a state of a cell in the culture tank model, wherein
the simulator execution unit causes an uncertainty in a state in accordance with the uncertainty parameter, to occur for the cell in the culture tank model.

12. The apparatus according to claim 3, further comprising:
an uncertainty parameter setting unit which sets, for a culture tank model which is included in the simulator, and which is a simulation model of a culture tank of the cell culture system, one or more uncertainty parameters of a state of a cell in the culture tank model, wherein
the simulator execution unit causes an uncertainty in a state in accordance with the uncertainty parameter, to occur for the cell in the culture tank model.

13. The apparatus according to claim 2, further comprising: a determination unit which determines whether a value indicating an execution result satisfies a predetermined condition, according to the simulator being executed.

14. The apparatus according to claim 3, further comprising: a determination unit which determines whether a value indicating an execution result satisfies a predetermined condition, according to the simulator being executed.

15. The apparatus according to claim 4, further comprising: a determination unit which determines whether a value indicating an execution result satisfies a predetermined condition, according to the simulator being executed.
